(19) United States
(12) Patent Application Publication
(10) Pub. No.: US 2014/0263558 A1
(43) Pub. Date: Sep. 18, 2014

(54) EXTENDED CURVED TIP FOR SURGICAL APPARATUS

(71) Applicant: Cardica, Inc., Redwood City, CA (US)

(72) Inventors: Bernard A. Hausen, Redwood City, CA (US); Bryan D. Knodel, Flagstaff, AZ (US)

(73) Assignee: Cardica, Inc., Redwood City, CA (US)

(21) Appl. No.: 14/206,886

(22) Filed: Mar. 12, 2014

Related U.S. Application Data
(60) Provisional application No. 61/791,795, filed on Mar. 15, 2013.

(51) Int. Cl. A61B 17/068

(52) U.S. Cl. CPC A61B 17/068
USPC 227/176.1; 227/175.1

(57) ABSTRACT

A surgical instrument is implemented with an extended curved tip to facilitate maneuvering and manipulating around small spaces and tissues at a surgical site in the body of a patient. The surgical instrument comprises of an end-effector and an extended curved tip that is coupled to the end-effector. A proximal portion of the extended curved tip is coupled to a distal portion of the extended curved tip, and a distal portion of the extended curved tip is configured to be a substantially tapered end or a tapered free-end.

### CROSS-REFERENCE TO RELATED APPLICATION(S)

The application claims priority to Provisional U.S. Patent Application No. 61/791,795 (Attorney Docket No. 366), filed on Mar. 15, 2013, which is incorporated herein by reference.

### FIELD OF THE INVENTION

The invention is generally related to surgical devices; more particularly, to surgical stapling devices.

### BACKGROUND

Surgical stapling devices, such as endocutters, typically staple and cut tissue to transect that tissue while leaving the cut ends of that tissue hemostatic. More advanced surgical stapling devices typically have end-effectors that are small enough in diameter so that they can be used in minimally invasive surgical procedures where access to a surgical site is obtained through a trocar, port, or small incision in the body of a patient.

### SUMMARY OF THE INVENTION

A surgical instrument is implemented with an extended curved tip to facilitate maneuvering and manipulating around small spaces and tissues at a surgical site in the body of a patient. The surgical instrument comprises of an end-effector and an extended curved tip that is coupled to the end-effector. A proximal portion of the extended curved tip is coupled to a distal portion of the extended curved tip, and a distal portion of the extended curved tip is configured to be a substantially tapered end or a tapered free-end.

In one embodiment, the distal portion of the end-effector that is coupled to the proximal portion of the extended curved tip is a staple holder. In another embodiment, the distal portion of the end-effector that is coupled to the proximal portion of the extended curved tip is a staple holder channel. In yet another embodiment, the surgical instrument may be a surgical stapling device. The surgical stapling device may include a removable staple cartridge to provide surgical staples to be deployed by the surgical stapling device and an anvil to deform the surgical staples. The extended curved tip may be coupled to the removable staple cartridge. In such an embodiment, the proximal portion of the extended curved tip may be coupled to a distal portion of the removable staple cartridge and a distal portion of the extended curved tip may be configured to be a substantially tapered end or a tapered free-end.

In regards to the extended curved tip, the substantially tapered end or tapered free-end of the distal portion may have a progressively tapering profile. In addition, the tapered end or tapered free-end may have a substantially rounded tip or point.

Further details about the extended curved tip, it may include a transition arch on a top surface of the extended curved tip. The transition arch may be located between the proximal portion and the distal portion of the extended curved tip. In addition, the extended curved tip may include a sliding-arch bottom on at least a portion of its bottom surface. The sliding-arch bottom may be located between the proximal portion and the distal portion of the extended curved tip.

More details about the extended curved tip, the extended curved tip may include an angular tilt along it body. The angular tilt may be oriented in a substantially upward direction or orientation in respect to a lateral axis. The lateral axis may be along the body of the end-effector of the surgical instrument. In some embodiments, the angular tilt of the body of the extended curved tip may be less than 30 degrees. In some embodiments, the angular tilt of the body of the extended curved tip may be more than 30 degrees.

### BRIEF DESCRIPTION OF THE DRAWINGS

FIG. 1 illustrates a surgical device with an extended curved tip.

FIG. 2A and FIG. 2B illustrate close-up views of surgical devices with examples extended curved tips.

FIG. 3A and FIG. 3B illustrate exposed isometric views of components of surgical devices coupled to two examples of extended curved tips.

FIG. 4A and FIG. 4B illustrate exposed side-views of components of surgical devices coupled to two examples of extended curved tips.

FIG. 5A-1 and FIG. 5A-2 illustrate isometric views of two examples of extended curved tips.

FIG. 5B-1 and FIG. 5B-2 illustrate side views of two examples of extended curved tips.

FIG. 5C-1 and FIG. 5C-2 illustrate front views of two examples of extended curved tips.

FIG. 5D-1 and FIG. 5D-2 illustrate top views of two examples of extended curved tips.

### DETAILED DESCRIPTION

Surgical stapling devices, such as endocutters, typically staple and cut tissue to transect that tissue while leaving the cut ends of that tissue hemostatic. More advanced surgical stapling devices typically have end-effectors that are small enough in diameter so that they can be used in minimally invasive surgical procedures where access to a surgical site is obtained through a trocar, port, or small incision in the body of a patient. Referring to FIG. 1, it illustrates a surgical device 100, such as a surgical stapling and cutting device, with an extended curved tip 500 which addresses some of the issues or difficulties involved with minimally invasive surgical procedures. One example of a surgical stapling and cutting device using an extended curved tip is the MicroCutter XChange® 30 that is designed and manufactured by Cardica, Inc. (“Cardica”) of Redwood City, Calif. As discovered by Cardica, it is particularly useful to implement an extended curved tip to a surgical instrument, such as the MicroCutter XChange® 30. An extended curved tip implemented at the distal end of a surgical instrument will allow for easier manipulation of the instrument in, around, and through tissue during surgical procedures. For purposes of illustration, Cardica discovered that it is particularly useful to implement the extended curved tip 500 to the bottom portion of the end-effector 200 of the surgical instrument 100, as illustrated in FIG. 2A and FIG. 2B. For example, the extended curved tip 500 can be implemented to a staple holder member of a stapling device—the staple device may be a cartridge-less staple device or a cartridge-based staple device. As one example, for a cartridge-less staple device, the extended curved tip 500 may be implemented to the structural component(s) of the staple holder. In another example, for a cartridge-based staple device, the extended curved tip 500 may be implemented to a removable staple cartridge. In a further example, the extended curved tip 500 may be implemented to the staple holder channel—a component of the staple device that holds the removable staple cartridge. In addition, for some applications, the extended curved tip 500 can also be implemented to the anvil member of a stapling device. For illustration purposes, without limiting the various implementations and applications of the extended curved tip, FIG. 2A and FIG. 2B illustrate the anvil member 250 being oriented on top of the staple holder member 260 of the surgical apparatus.

For purposes of further illustration, FIG. 3A and FIG. 3B illustrate exposed isometric views of component 300 of the surgical device coupled to the extended curved tips 500. As illustrated, the extended curved tip 500 can be coupled to the distal portion of an end-effector of a surgical apparatus in any suitable manner. For example, the extended curved tip 500 can be attached, press-fitted, injection molded, overmolded, glued, fastened, etc. onto one or more components or members of an end-effector of a surgical device. Overmolding is a molding process in which two or more materials or parts are combined to produce a single part. For example, FIG. 4A and FIG. 4B illustrate exposed side views of component 300 of the surgical device coupled to the extended curved tips 500 by way of one of the processes mentioned.

As can be seen in FIGS. 3A through 3B and FIGS. 4A through 4B, the extended curved tips 500 extend and curve substantially in an upwardly direction. As can be appreciated from the various Figures, the extended curved tip may embody various different designs and constructions. For example, an upwardly tilt of the extended curve tip may be constructed with different tilt angles. As in one embodiment, illustrated in FIG. 4A, the tilt angle of the distal tip may be small than the tilt angle of another embodiment, illustrated in FIG. 4B. In some embodiments, the tilt angle of the distal tip of the extended curved tip 500 may be less than 30 degrees about an axis of the end-effector 200. In some other embodiments, the tilt angle of the distal tip may be greater than 30 degrees about the axis of the end-effector 200. In one embodiment, the tilt angle of the distal tip may be about 45 degrees about the axis of the end-effector. In another embodiment, the tilt angle of the distal tip may be about 60 degree about the axis of the end-effector.

Details of the extended curved tip 500 are further illustrated and described along with the isometric views of the extended curved tip 500, as provided from FIG. 5A-1 & FIG. 5A-2 through FIG. 5D-1 & FIG. 5D-2. In the illustrated configurations, the substantially upwardly tilt of the distal tip allows for easier manipulation of the tip portion 510 of the surgical device in, around, and through tissue. The substantially upward angle of the tip 510 may provide some mechanical advantage as the tip is manipulated in, around, and through tissue—as well as maneuvering the tip in, around, through tight spaces, openings, gaps, etc. It should be noted that the extended curved tip 500 has a substantially rounded and tapered tip 510. The roundness of the tip 510 prevents the likelihood of puncturing or piercing tissue; however, it does allow for getting in, around, and through tight spaces, openings, gaps, etc., much easier than a non-tapered tip. In addition, the extended curved tip or the shape of the tip may be progressively tapered to a substantially rounded point at the distal end of the extended tip 510. As such, the progressively tapered shape of the extended tip has inherently greater structural strength, substantially greater rigidity, less likely to bending, etc., which makes this extended tip 500 much more suitable for working or moving around tissues, tight spaces, etc. This tapered profile is shown more clearly in FIG. 5A-1 & FIG. 5A-1 through FIG. 5D-1 & FIG. 5D-2.

Also, as can be seen in FIG. 4A and FIG. 4B, based on the side or profile view, the extended curved tip 500 has a progressively tapered thickness. This is further illustrated in FIG. 5B-1 & FIG. 5B-2. That is, the extended curved tip 500 is substantially thicker toward the proximal portion, while substantially thinner or tapered forward the distal portion of the tip 510. This tapered construction further allows for greater strength, stiffness, and maneuverability in, around, and through tissue. A transition arch 520, as illustrated in FIG. 5A-1 and FIG. 5A-2, between the distal portion and proximal portion of the extended curved tip 500. The transition arch 520 also provides for greater ease of transition in, around, and through tissue as the end-effector of the surgical device is manipulated by a surgeon during a surgical procedure. For example, tissue may smoothly translate or move across the transition arch 520, without catching or binding on the extended curved tip 500 or the end-effector 200 of the surgical device 100. In addition, the transition arch may make the extended tip much a stronger structure or element, than one without a transition arch 520. As can be appreciated, the extended curved tip 500 may be incorporated to any surgical device. Its use and advantages are not limited to surgical stapling devices.

Also illustrated in FIG. 4A & FIG. 4B and FIG. 5B-1 & FIG. 5B-2, the extended curved tip 500 has a substantially sliding-arch bottom 530 that facilitates or provides a smooth transition for translating around, across, or through tissue. The sliding-arch bottom has an angular tilt facilitate a smooth transition for translating around, across, or through tissue. The shape, construction, etc. of the extended curved tips are all designed or optimized to assist with or facilitate movement of the distal portion of an end-effector of a surgical instrument to maneuver about tight spaces, tightly packed organs or tissues, etc.

As illustrated in FIG. 5C-1 & FIG. 5C-2, the geometry of the extended curved tip 500 is optimized such that the tip along with the component(s) that it is attached to can easily fit into and through a substantially small opening. This feature can be of particular importance. For example, some of the surgical devices using the extended curved tip 500 may be used in minimally invasive surgical procedures. As such, the operational portion of the surgical device, such as an end-effector, must fit through a small port, trocar, or opening, which may be about 5 mm in diameter. Often times, the small port, trocar, or opening may not be expandable. That is, the small port, trocar, or opening cannot be expanded to a larger opening; as such, it is very important that the operational portion, more particularly the entry or tip portion, of the surgical device be small enough to fit through the small port, trocar, or opening. As FIG. 5C-1 & FIG. 5C-2 illustrate the frontal profiles of the extended curved tips, the maximum diameters of the extended curved tips 500 are significantly small, and for most embodiments, maximum (frontal) diameters are either substantially the same or substantially smaller than the maximum diameter of the end-effector 200 of the surgical instrument 100.

While the details of the invention have been described, it will be apparent to one skilled in the art that various changes and modifications can be made and equivalents employed, without departing from the present invention. It is to be understood that the invention is not limited to the details of construction, the arrangements of components, and/or the method set forth in the above description or illustrated in the drawings. Statements in this disclosure are merely exemplary; they are not, and cannot be interpreted as, limiting the scope of the claims. Further, the figures are merely exemplary and are also not limiting. Therefore, the details of the invention are not to be restricted or limited; instead, they are to be interpreted in accordance with the following claims and their equivalents.

What is claimed is:

1. A surgical device, comprising:
an end-effector;
an extended curved tip coupled to the end-effector, wherein a proximal portion of the extended curved tip configured to couple to a distal portion of the end-effector and a distal portion of the extended curved tip configured to be a substantially tapered end or a tapered free-end.

2. The surgical device of claim 1, wherein the distal portion of the end-effector coupled to the proximal portion of the extended curved tip is a staple holder.

3. The surgical device of claim 1, wherein the distal portion of the end-effector coupled to the proximal portion of the extended curved tip is a staple holder channel.

4. The surgical device of claim 1, wherein the substantially tapered end or tapered free-end of the distal portion of the extended curved tip has a progressively tapering profile.

5. The surgical device of claim 1, wherein the substantially tapered end or tapered free-end of the distal portion of the extended curved tip has a substantially rounded tip or point.

6. The surgical device of claim 1, wherein the extended curve tip includes a transition arch on a top surface.

7. The surgical device of claim 6, wherein the transition arch configured to be located between the proximal portion and the distal portion of the extended curved tip.

8. The surgical device of claim 1, wherein the extended curved tip included a sliding-arch bottom on a bottom surface.

9. The surgical device of claim 8, wherein the sliding-arch bottom configured to be located between the proximal portion and the distal portion of the extended curved tip.

10. The surgical device of claim 1, wherein the extended curved tip configured with an angular tilt.

11. The surgical device of claim 10, wherein the angular tilt is at a substantially upward direction or orientation in respect to a lateral axis of the end-effector.

12. The surgical device of claim 10, wherein the angular tilt is less than 30 degrees in respect to a lateral axis of the end-effector.

13. The surgical device of claim 10, wherein the angular tilt is greater than 30 degrees in respect to a lateral axis of the end-effector.

14. The surgical device of claim 10, wherein the angular tilt is about 30 degrees in respect to a lateral axis of the end-effector.

15. The surgical device of claim 10, wherein the angular tilt is about 45 degrees in respect to a lateral axis of the end-effector.

16. The surgical device of claim 10, wherein the angular tilt is about 60 degrees in respect to a lateral axis of the end-effector.

17. A surgical stapling device, comprising:
a removable staple cartridge to provide surgical staples to be deployed by the surgical stapling device;
an anvil to deform the surgical staples as the surgical staples are deployed by the surgical stapling device;
an extended curved tip coupled to the removable staple cartridge, wherein a proximal portion of the extended curved tip configured to couple to a distal portion of the removable staple cartridge and a distal portion of the extended curved tip configured to be a substantially tapered end or a tapered free-end.
